(19) United States
(12) Patent Application Publication
(10) Pub. No.: US 2023/0330389 A1
(43) Pub. Date: Oct. 19, 2023

(54) VARIABLE STIFFNESS MULTI-LUMEN TUBE

(71) Applicant: Boston Scientific Scimed, Inc., Maple Grove, MN (US)

(72) Inventors: Gene Thomas STORBECK, Millis, MA (US); Man Minh NGUYEN, Harvard, MA (US)

(73) Assignee: Boston Scientific Scimed, Inc., Maple Grove, MN (US)

(21) Appl. No.: 18/300,966

(22) Filed: Apr. 14, 2023

Related U.S. Application Data
(60) Provisional application No. 63/332,321, filed on Apr. 19, 2022.

(51) Int. Cl. A61M 25/00; A61M 25/01

(52) U.S. Cl. CPC A61M 25/0053; A61M 25/0147; A61M 25/0026; A61M 25/0009

(57) ABSTRACT

A medical device includes a shaft. The shaft includes an integral tube defining a plurality of lumens. The integral tube is comprised of at least one of PTFE and ePTFE. A density of the at least one of PTFE and ePTFE varies along a length of the integral tube.

### CROSS-REFERENCE TO RELATED APPLICATIONS

This application claims the benefit of priority under 35 U.S.C. § 119 from U.S. Provisional Application No. 63/332,321, filed Apr. 19, 2022, which is incorporated by reference herein in its entirety.

### TECHNICAL FIELD

The disclosure relates generally to medical devices, systems, and related methods having multi-lumen tubes of polytetrafluoroethylene (PTFE) and/or expanded polytetrafluoroethylene (ePTFE), where regions of the tubes have differing stiffness, density, and/or other characteristics.

### BACKGROUND

Medical devices used during endoscopic or other procedures, e.g., catheters, endoscopes, sheaths, tubes, and the like, require a set of properties that allow the devices to function as intended. For example, during procedures such as endoscopy, colonoscopy, and/or endoscopic retrograde cholangiopancreatogram (“ERCP”), an operator may insert a medical device into a patient, and guide that medical device across tortuous anatomy for positioning the device at target site deep in the body. Access to the target site may require articulation or movement of the distal portion of the device, as well as bending of more proximal portions of the device, to efficiently navigate to the target site and direct the distal end at the site. The medical device may therefore require portions with varying characteristics, such as differing flexibilities/stiffnesses.

### SUMMARY

A medical device may comprise a shaft. The shaft may include an integral tube defining a plurality of lumens, the integral tube comprised of at least one of PTFE and ePTFE, and wherein a density of the at least one of PTFE and ePTFE varies along a length of the integral tube. The density may vary within a cross-section of the integral tube. In alternative embodiments, a density of a radially inner portion of the integral tube may differ from a density of a radially outer portion of the integral tube. The integral tube may include an articulation joint. At least two regions separated about a circumference of the articulation joint may have densities less than other regions about the circumference. The at least two regions may be separated about the circumference by approximately 180 degrees. In alternative embodiments, the integral tube comprises an articulation joint, and the integral tube further includes a portion proximal to the articulation joint, wherein the articulation joint is less dense than the proximal portion. In some embodiments, the device may further comprise one of a braid or a coil surrounding an outermost surface of the integral tube. The one of the braid or the coil may be heated to a temperature greater than or equal to 250 degrees Celsius. At least one of a pitch, a material, and a thickness of a wire of the one of the braid and the coil may vary along a length of the one of the braid and the coil. The integral tube further may further comprise at least one mandrel extending through at least one of the plurality of lumens. The at least one mandrel may be at a temperature greater than or equal to 250 degrees Celsius. In alternative embodiments, integral tube may further comprise a plurality of mandrels extending through more than one lumen of the plurality of lumens. A first mandrel of the plurality of mandrels may be at a first temperature, and a second mandrel of the plurality of mandrels may be at a second temperature different from the first temperature. A density of a proximal portion of the integral tube may differ from a density of a distal portion of the integral tube. The medical device may be an endoscope, the shaft may be connected to a handle at a proximal end of the shaft, and the plurality of lumens may include a working channel and lumens for articulation wires.

A method of fabricating a tube may comprise stretching a heated, integral starting tube of at least one of PTFE and ePTFE, to result in an intermediate tube having a density less than a density of the integral starting tube, wherein the starting tube defines multiple lumens; and linearly retracting only a first portion of a length of the intermediate tube when the intermediate tube is heated, to result in a final tube, wherein the first portion has a greater density than a second portion of the final tube. The method may further comprise inserting a mandrel through a lumen of the multiple lumens; heating the mandrel to greater than or equal to 250 degrees Celsius; and removing the mandrel. Tension may be applied to at least one of the starting tube, the intermediate tube, and the final tube. The linear retraction may be controlled by applying a force in a longitudinal direction of the tube.

A method of forming or fabricating a tube may further include inserting at least one mandrel through at least one lumen of a multi-lumen tube comprising at least one of PTFE and ePTFE; heating the mandrel to a temperature of at least 250 degrees Celsius to result in a first region of the tube having a higher density than a second region of the tube, the first region and the second region being in a same cross-section of the tube; and removing the at least one mandrel.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 depicts an exemplary medical device, according to aspects of this disclosure.

FIG. 2 depicts an exemplary method of manufacture for a PTFE tube, according to aspects of this disclosure.

FIGS. 3A-B depict a tube in a straight configuration (FIG. 3A) and in a bent configuration (FIG. 3B), according to aspects of this disclosure.

FIG. 4 depicts an exemplary section of a multi-lumen tube with variable stiffness, according to aspects of this disclosure.

FIGS. 5A-B depict an exemplary method of manufacture for a tube with variable stiffness (FIG. 5A) and an exemplary depiction of the resulting tube (FIG. 5B), according to aspects of this disclosure.

FIGS. 6A-B depict an alternative exemplary method of manufacture for a tube with variable stiffness (FIG. 6A) and an exemplary depiction of the resulting tube (FIG. 6B), according to aspects of this disclosure.

FIGS. 7A-E depict exemplary cross sections of a multi-lumen tube with varying density, according to aspects of this disclosure.

FIG. 8 depicts a distal end portion of an exemplary multi-lumen tube with variable stiffness, according to aspects of this disclosure.

### DETAILED DESCRIPTION

Reference will now be made in detail to aspects of the disclosure, examples of which are illustrated in the accompanying drawings. Wherever possible, the same or similar reference numbers will be used through the drawings to refer to the same or like parts. The term “distal” refers to a portion farthest away from a user when introducing a device into a subject (e.g., a patient). By contrast, the term “proximal” refers to a portion closest to the user when placing the device into the subject.

Embodiments of the disclosure may solve one or more of the limitations in the art. The scope of the disclosure, however, is defined by the attached claims and not the ability to solve a specific problem. The disclosure, in certain embodiments, is drawn to medical devices including at least one multi-lumen feature (e.g., a tube) having at least two portions of variable stiffness or flexibility. Although the disclosure may refer at different points to one of a duodenoscope or an endoscope, it will be appreciated that, unless otherwise specified, duendoscopes, endoscopes, colonoscopes, ureteroscopes, bronchoscopes, laparoscopes, sheaths, catheters, grafts, tubes, sheaths, any suitable delivery device or other medical device may be used in connection with aspects of this disclosure.

Portions of a medical device that may have a stiffness differing from other portions or a remainder of the medical device may include any structure requiring such differing stiffness. For example, a tube may include an axial portion, such as a distal portion, having greater flexibility than remaining portions of the tube or shaft. This may allow, for example, a distal articulation joint to be more flexible than the more proximal portions of the tube, allowing for easier distal steering. In another example, one or more portions of a cross-section of a tube may have a different flexibility than other portions of the cross-section. This may allow a tube to bend more easily in certain planes where the cross-section is most flexible, for more efficient steerability of the tube. In another example, at least one lumen of the multi-lumen tube may have walls of greater flexibility than walls defining other lumens of the tube. This may allow that at least one lumen to perform its function more effectively, for example delivery of working tools, cables, fluid, or the like. In still further examples, any variable stiffness portion of a tube may be used in any suitable combination with any other variable stiffness portion of a tube, e.g. an axial distal portion has differing flexibility from more proximal portions and/or variable stiffness exists within cross-sectional portions of the tube.

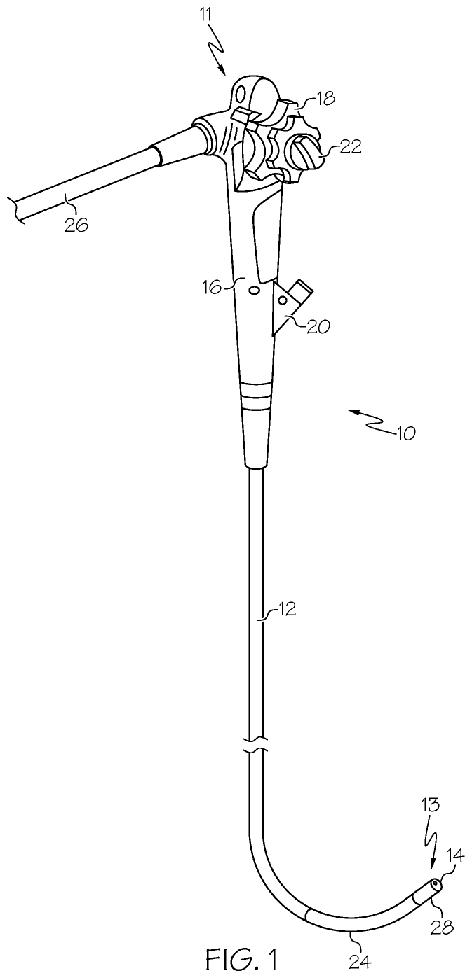

FIG. 1 shows an exemplary medical device 10 (e.g. an endoscope) in accordance with an embodiment of this disclosure. Medical device 10 includes a proximal end 11 and a distal end 13. A handle 16, with one or more knobs 18, locks 22, and ports 20, is at or near proximal end 11. A shaft 12 extends from a distal end of a handle 16 to a distal end 13 of device 10.

Shaft 12 is a tube having sufficient length and flexibility to access sites within the body and traverse tortuous anatomy. Shaft 12 may be a single component with one or multiple lumens. Alternatively, shaft 12 may be comprised of multiple components, for example, multiple tubes with single or multiple lumens connected together. A plurality of lumens 14 may extend through shaft 12 from the proximal end to the distal end 13. The plurality of lumens 14 may comprise any number of lumens desired and as permitted given the constraints associated with the cross sectional area and desired characteristics of shaft 12. For example, lumens 14 may include lumens for insertion/delivery of instruments, delivery of fluid, application of suction, housing of electrical cables or wires, housing of wires for steering (connecting the handle to a distal portion), etc. One or more of the plurality of lumens 14 may be open at a distal face of shaft 12, as shown in FIG. 1, for example to deliver fluid or an instrument distal to the end of device 10. In addition, or alternatively, one or more of the plurality of lumens 14 may terminate at a position proximal to the distal face of shaft 12, or a cap 28 may be placed at a distal end of shaft 12 to close the end of one or more of the plurality of lumens 14, for example to enclose electrical cabling for imaging and lighting. Each of the plurality of lumens 14 may be substantially parallel to each other, so that longitudinal axes of the plurality of lumens 14 are substantially parallel to one another and to the longitudinal axis of the shaft 12. The plurality of lumens 14 may comprise a variety of shapes (e.g. square, oval, circular, star, etc.) and sizes. Shaft 12 is connected to handle 16 at the proximal end of shaft 12, permitting access to one or more of the plurality of lumens 14 from the one or more ports 20 through handle 16. Additionally or alternatively, the plurality of lumens 14 may terminate inside the handle 16 and communicate with structure within the handle and/or an umbilicus 26, for suction, irrigation, electronics, etc., as is known in the art.

Medical device 10 may further include an articulation portion 24 for navigating tortuous anatomy and directing the distalmost end of device 10 towards a target site. Articulation portion 24 may be located at or just proximal to distal end 13 of shaft 12. Articulation portion 24 may enable a user to bend or articulate the distal end 13 in any desired direction (e.g., up, down, left, and/or right) through known means in the art, particularly through the manipulation of knobs 18 and control wires (not shown) contained within the handle 16 and lumens 14 of shaft 12. The articulation portion 24 may be one portion of an integral, one-piece shaft 12. For example, shaft 12 may comprise a section of sufficient axial length having the materials and flexibility characteristics described further herein. In an alternative embodiment, articulation portion 24 may be a separate component coupled to a distal end of shaft 12. The cap 28 may be coupled to a distalmost end of articulation portion 24 in either embodiment.

The entirety of shaft 12 and/or one or more portions of shaft 12 (e.g., articulation joint 24) may include a tube comprised of polytetrafluoroethylene (PTFE) or expanded polytetrafluoroethylene (ePTFE). PTFE and ePTFE are materials commonly used in the medical device industry due to their various biocompatibility and mechanical properties.

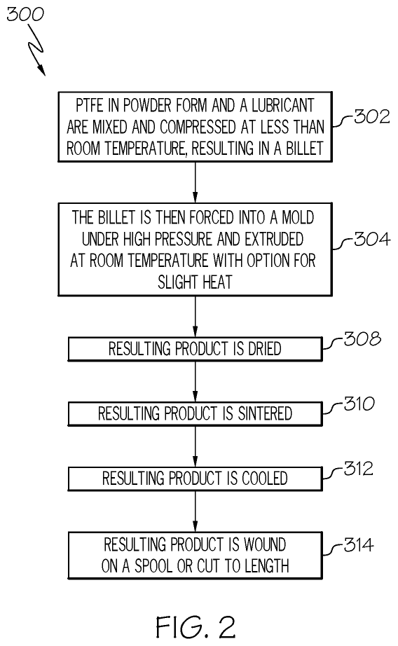

PTFE tubes may be manufactured using a variety of techniques commonly known in the art. Primarily, PTFE tubes are paste or ram extruded. FIG. 2 is a flow diagram of an exemplary paste extrusion process 300. In a first step 302 of process 300, PTFE in powder form and a lubricant are mixed then compressed at less than room temperature resulting in a billet.

One or more types of PTFE in powder form may be used in the billet. For example, the billet may be comprised of a single type of PTFE in powder form, or the billet may be comprised of different types of PTFE in powder form. For example, the billet may be comprised of two or more layers of different types of PTFE in powder form. Each of the layers may be a same depth or thickness, or the layers may be of different depths or thicknesses.

In further examples, a cross-section of the billet may be comprised of a single type of PTFE in powder form, or of different types of PTFE in powder form. For example, a first portion of the cross-section of the billet may be comprised of a first type of PTFE in powder form, and a second portion of the cross section of the billet may be comprised of a second type of PTFE in powder form. The first, second, etc. portions of the cross section may be the same size or a different size.

For example, the cross-section may be divided into two halves, three thirds, four quadrants, etc. Each half, third, quadrant, etc. may be the same size and/or shape or different sizes and/or shapes. For example, if the billet's cross-section is generally circular, a first quadrant may be approximately 90 degrees, a second quadrant may be approximately 90 degrees, etc. Additionally or alternatively, the quadrants may be different degrees. In such a way, the cross section of the billet may be symmetric or asymmetric. Accordingly, the resulting tube may have symmetric or asymmetric characteristics. A tube having asymmetric characteristics may help, for example, to create a tube having one or more portions with greater sealing capabilities and/or greater flexibility.

In still further examples, the billet may be comprised of different layers and of different portions of PTFE in powder form. For example, a first longitudinal length of the billet may be comprised of different layers of PTFE in powder form, and a second longitudinal length of the billet may be comprised of different cross-sectional portions of PTFE in powder form. As will be described in further detail below, the different types of PTFE in powder form may be utilized to help impart different properties or characteristics of the manufactured tube, for example, along the longitudinal length or radial width of the manufactured tube.

The billet is then forced into a mold under high pressure and extruded at room temperature, or with slight heating (step 304). According to embodiments of this disclosure, any desired PFTE resin commonly known in the art may be used to manufacture a tube, depending on the desired properties of the resulting device using the tube. Furthermore, lubricants or alternative additives known in the art may be added to the resin, as necessary to achieve the desired characteristics of the tube and device. Similar to the PTFE in powder form discussed previously, the lubricants or alternative additives may be added in different layers or in different cross-sectional portions of the billet. The resulting tube is then subjected to drying processes to remove the lubricant. At this point, the tube is commonly referred to as a “green PTFE/ePTFE tube.” The tube then undergoes sintering and cooling processes (steps 308, 310, 312). During the sintering step 310, the tube may undergo controlled tensioning or stretching processes to minimize post-extrusion stretching. Sintering, which involves heating the tube, allows coalescence of the PTFE resin particles, which provides strength and void reduction within the tube. The end product is a fully sintered PTFE tube commonly wound up on a spool or cut to a desired length (step 314).

ePTFE is a microporous version of PTFE, resulting in different mechanical properties. ePTFE, for example, may be softer and more flexible than PTFE. The manufacturing or fabricating process is similar to that of PTFE with a few differences. Specifically, the PTFE tube is subjected to additional manufacturing or fabricating processes after the extruding and drying processes stated above. These additional processes include subjecting the tube to longitudinal and transverse expansion processes at a specific rate and elevated temperatures, resulting in an amorphously locked ePTFE tube.

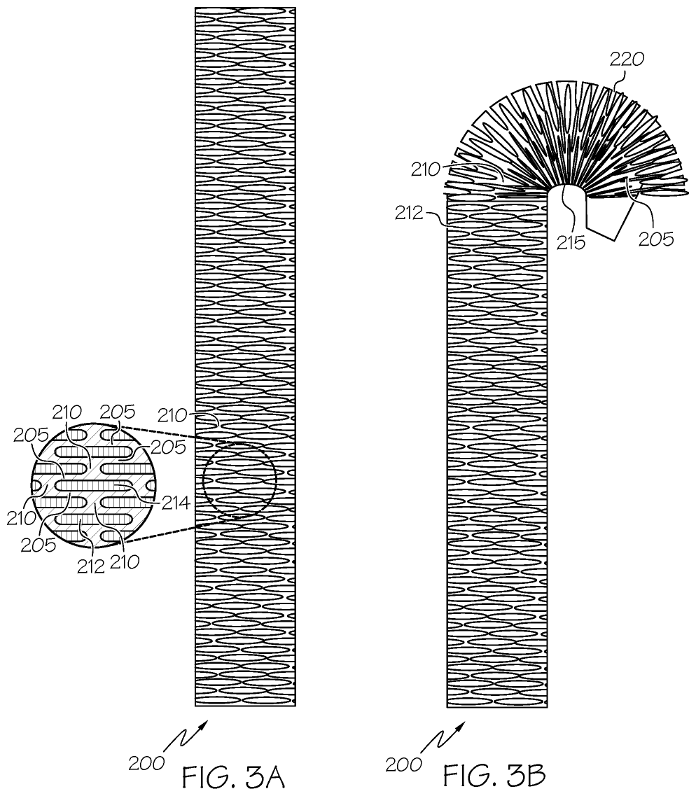

ePTFE is used in medical applications, including as tubing, for a variety of reasons. For example, an ePTFE tube is able to bend without kinking. This is due to the fibril and nodal microstructure of ePTFE. FIG. 3A shows an ePTFE tube 200, having an exemplary fibril and nodal microstructure, in a straight configuration. The microstructure of tube 200 includes roughly parallel-running clumps of material (nodes 205) with perpendicular fibers (called fibrils 210) connecting them. Pores 212 exist within the network of nodes 205 and fibrils 210. Pores 212 also include fibrils 214 oriented in an axial direction. FIG. 3B shows tube 200 in a bent configuration. During bending, at an inner arc 215, nodes 205 will compress pores 212 and fibrils 210, 214 will compress. Along an outer arc 220, fibrils 210, 214 are placed under tension, and pores 212 increase in size. In combination, this is known as foreshortening, which enables tube 200 to maintain its cross-sectional shape when bent. For example, lumens within tube 200 will substantially retain their shape despite bending, making ePTFE tubes particularly well-suited for use as shafts and tubes in various medical applications.

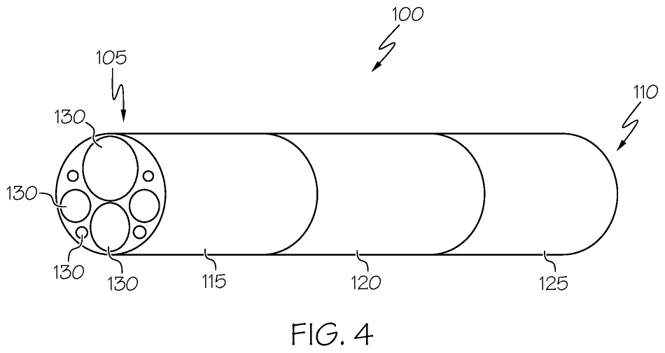

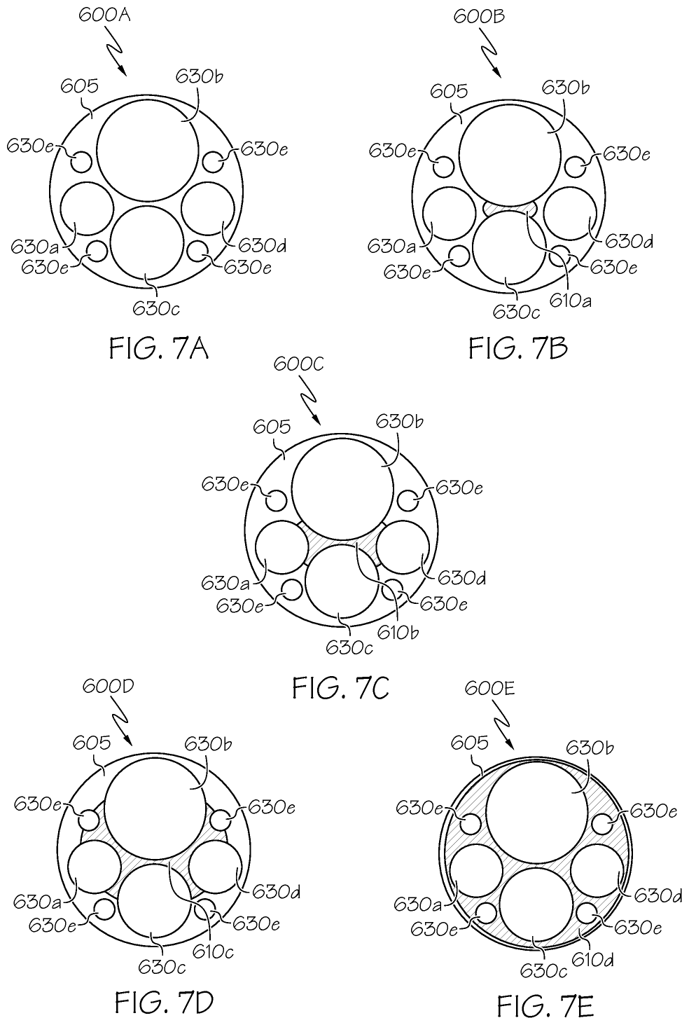

As mentioned above, various medical applications require a multi-lumen tube. FIG. 4 shows a tube 100 with a plurality of lumens 130 extending from a proximal end 105 to a distal end 110. The tube may have one or more discrete axial portions 115, 120, 125 of varying density and flexibility. For example, portion 115 may be a portion of high density and low flexibility, with portions 120, 125 of decreasing densities and increasing flexibilities, or vice versa. Alternatively, portions 115, 125 may be portions of higher density and lower flexibility relative to portion 120, or vice versa. The varying density and flexibility of portions 115, 120, and 125 may be achieved by, for example, layering and/or portioning the billet (not shown) from which tube 100 is extruded, as discussed above. Additionally or alternatively, tube 100 may be subjected to additional process for creating portions of PTFE and ePTFE tubes of varying density and flexibility, as will be discussed below. The number of axial portions are not limited to three, as shown. For example, there can be more or less axial portions of differing densities. Furthermore, the number of lumens 130 is not limited to the shown configuration. There can be more or less lumens, and their sizes may vary. Additional multi-lumen tubes according to embodiments of this disclosure will be described in more detail in FIGS. 7A to 7E.

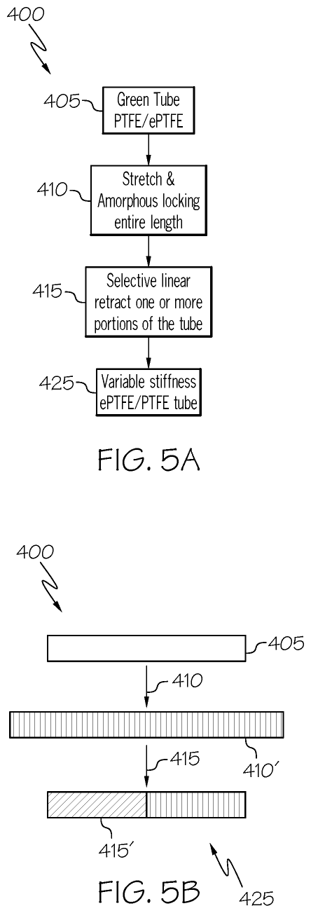

Embodiments of this disclosure relate to processes for creating portions of PTFE and ePTFE tubes of varying density and flexibility. In one example, depicted in FIGS. 5A and 5B, the process 400 begins with a green PTFE or ePTFE tube 405 (e.g., a starting tube), such as a multi-lumen tube 405. The entire length of the multi-lumen tube 405 is stretched and amorphously locked (step 410), resulting in a longer, intermediate tube 410′ of decreased density and increased flexibility along the entire intermediate tube 410′. Alternatively, to stretch and amorphously lock green tube 405, the tube 405 can be partially sintered and amorphously locked at a lower temperature or subjected to a higher or lower stretching rate. Subsequently, sections can be retracted to a different sintering level. However, in exemplary embodiments, once the tube is in a fully sintered state or heated up to or near the melting temperature, the tube is not further modified in this step 410.

In the next step 415, a portion of intermediate tube 410′ is heated to a high temperature to a level up to and including full sinter, while remaining portions of intermediate tube 410′ are not heated. For example, an axial portion on the left hand side of intermediate tube 410′ may be heated to result in a portion 415′ of a final tube 425 being more dense and less flexible than the remaining portions of final tube 425. This densification process results in linear retraction of the heated portion of intermediate tube 410′, i.e. the axial length of portion 415′ is shortened by the heating.

The degree of linear retraction can be controlled by applying a force, for example tension, to intermediate tube 410′ in a longitudinal direction of the tube during this heating step 415. This may be accomplished by, for example, applying weights to intermediate tube 410′. This results in a variable stiffness tube, e.g., final tube 425, where one portion 415′ has been selectively linear retracted (higher density and less flexibility), and another portion has been stretched and amorphously locked (lesser density and more flexibility). The optimal temperature used in step 415 may vary depending on the desired characteristics of the part of the tube. For example, the part may be heated to a temperature of approximately about 350-370 degrees Celsius for periods of five seconds to over one hour. However, the part may also be heated to a temperature of approximately about 250-450 degrees Celsius for a period between one second and several hours. For example, the part may be heated to a temperature greater than or equal to 250 degrees Celsius. Specifically, increasing the temperature may decrease the amount of time the part needs to be heated. Step 415 is different from the standard heating or sintering step 310 (FIG. 2) in that the part is first partially sintered or amorphously locked at a temperature lower than the sintering temperature. The tube is also subjected to stretching. Sections of the tube are then brought to a temperature above the prior temperatures. To restrict retraction of the sections, a force is applied longitudinally and heat is only applied to the areas for increased density. Radial compression may also be applied by means of a split interference mold, wherein the mold compresses the tube to help prevent the tube from recoiling.

The amount of linear retraction (of the heated portion in step 415) can be controlled in a variety of additional ways. For example, a braided or knitted wire tube may be placed firmly around the outside of intermediate tube 410′, prior to heating in step 415. A tube made of wire braiding or knitting has inherent compressive and expansion limits based on a variety of characteristics, including the size and material of the wires, the thickness of the wires, the pitch/density of the braid, the braid's contact pressure against intermediate tube 410′, etc. The tube may also vary in any of the previously mentioned characteristics along a longitudinal length. For example, the pitch/density of the braid may differ at a proximal portion than a distal portion. Linear retraction of intermediate tube 410′ may be controlled/limited by heating intermediate tube 410′ while part or all of intermediate tube 410′ is surrounded by the braid. The braid may be applied at any step after extrusion of the desired shape and drying of the part. However, it may be optimal to apply the braid after partial sintering or amorphously locking, as there is more structure at these steps.

Similarly, a metal coil may be wrapped around intermediate tube 410′, prior to heating in step 415. A coil has inherent compressive and expansion limits based on a variety of characteristics, including the size and material of the coil, the pitch of the coil, the coil's contact pressure against intermediate tube 410′, etc. Linear retraction of intermediate tube 410′ may be controlled/limited by heating intermediate tube 410′ while part or all of intermediate tube 410′ is surrounded by the coil. Similar to the braid or knitting pattern, the coil can be applied at any step after extrusion of the desired shape and drying of the part. However, it may be optimal to apply the coil after partial sintering or amorphously locking the part, as there is more structure at these steps.

In certain embodiments, by utilizing the braid, coil, or knitting pattern during step 415, localized energy around the metal braid or coil will impart localized heating and amorphous locking in adjacent regions of the ePTFE tube. This can impart a three-dimensional pattern of variable density and variable flexibility in the resulting ePTFE tube, the pattern substantially matching the braid or coil pattern. The patterns may improve the bonding capabilities of the part's surface because the surface area is much higher relative to a smooth tube. These features may also impart radial differences that could improve the hoop strength and modify the bending characteristics of the tube.

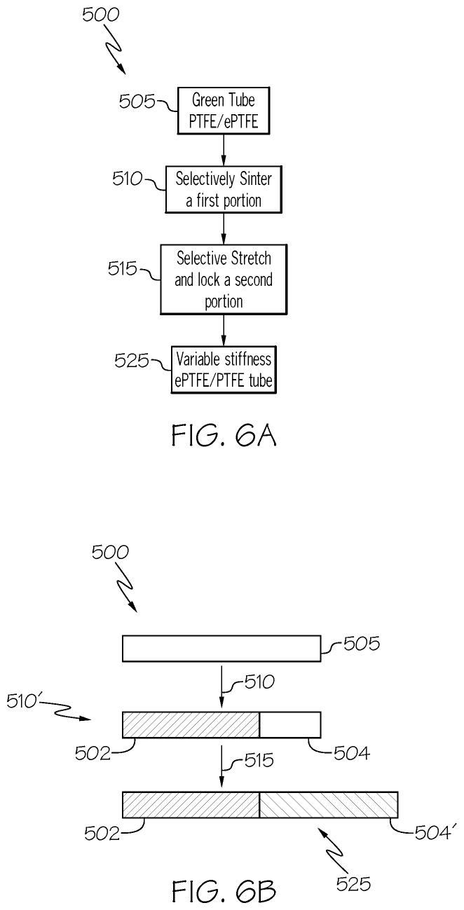

In an alternative process 500, depicted in FIGS. 6A and 6B, the process begins with a green PTFE or ePTFE tube 505, such as a multi-lumen tube 505. In step 510, a first axial portion of the green tube 505 may be heated to a fully sintered state, thereby increasing that portion's density and decreasing that portion's flexibility. The intermediate tube 510′ resulting from step 510 thereby includes a portion 502 of greater density and lesser flexibility than a remaining portion 504 of intermediate tube 510′. The sintered portion of intermediate tube 510′ may retract or shrink due to the sintering process.

In subsequent step 515, a portion of intermediate tube 510′, such as the entire portion 504 that was not heated in step 510, may be stretched and amorphously locked, thereby decreasing the density and increasing the flexibility in portion 504. Different temperatures, times, and/or tension can be applied to the tube to achieve different levels of sintering. Step 515 results in a final tube 525 with one portion 502 (the portion on the left hand side in FIG. 6B) being more dense and less flexible than another portion 504′ (the portion on the right hand side in FIG. 6B). The first and second portions 502, 504′ may be adjacent to one another (as in the FIGs.) or they may be spaced apart. Each portion 502, 504′ may be sintered in its corresponding process step 510, 515 to a different temperature, thereby creating a tube of variable densities and flexibilities. Similarly, each portion 502, 504′ may be stretched under different degrees of tension, which will alter the length, density, and flexibility of the corresponding portion.

In an alternative method of manufacture (not shown), the entire tube can be stretched at a first temperature by a first stretching rate. Subsequently, portions of the tube can be stretched further at a second temperature different from the first temperature and/or subjected to a second stretching rate different from the first stretching rate. This results in a tube with variable densities and characteristics, similar to the resulting tubes described above.

Certain challenges may arise in manufacturing or fabricating a multi-lumen tube with varying densities and flexibilities, particularly an ePTFE multi-lumen tube having the desired variations in density and flexibility. The large air gaps of the tube's lumens may cause variations in the conductance of heat to walls throughout the cross-section of the tube. For example, when applying heat from external the tube to the outer surface of the tube, the innermost walls defining lumens may not heat to the same temperatures as the outer walls, due to the large air gaps of the channels within the tube. This may cause variable densities and flexibilities across the cross section of the tube, with the outermost walls being the most dense and least flexible, and the innermost walls being relatively less dense and more flexible. Though this may have some desirable qualities, such as an inner lumen being more flexible for a particular function, it may result in a tube that does not have uniform foreshortening when the tube is bent, as described in connection with FIGS. 3A and 3B. To maintain this symmetrical and uniform bending through a multi-lumen structure, uniform (or substantially uniform) density throughout the cross-section may be desirable.

Conductance of heat to inner portions of the multi-lumen tube may be achieved by inserting mandrels within the lumens during the heating/sintering/amorphous locking steps to impart the desired heat conductance and achieve the desired cross-sectional pattern of densities and flexibilities, whether uniform or non-uniform.

The length(s) of the mandrels can also create density/flexibility differentials in the longitudinal, axial direction. The mandrels may be solid rods made of metal having diameters matching the diameters of the lumens in which they are inserted. When using heat external to the tube to heat the tube, the metal rod mandrels will conduct that heat into more central regions of the cross-section of the tube, resulting in a more uniform cross-sectional density and flexibility. Additionally or alternatively, the mandrels can be independently heated using a heat source directly coupled to the mandrels (e.g. at an exposed end of the mandrel), to add additional temperature to inner parts of the tube. Each mandrel may be independently heated, or all mandrels may be heated together.

Heating mandrels independently, at different temperatures, also may achieve a desired pattern of varying densities and flexibilities across a cross section of the multi-lumen tube. The cross-sectional density and flexibility of a tube may be controlled via induction heating mandrels inserted through the lumens of the tube. For example, mandrels may be strategically and selectively placed inside one or more lumens and inductively heated to suitable temperatures to create regions of varying density. This permits control over the density and flexibility in the tube's walls adjacent to and surrounding the lumens. In addition, using mandrels of different metals comprising differing metallurgical properties, including differing conductances, can be used to heat surrounding walls to desired temperatures. In some embodiments, the one or more mandrel may be heated to a temperature greater than or equal to approximately 250 degrees Celsius.

Additionally or alternatively, application of heat to outer portions of a single-lumen tube or the multi-lumen tube (e.g., flexible tube) may be achieved by partially or completely surrounding an outer surface of the flexible tube with a hollow tube. Depending on the desired density/flexibility differentials or characteristics of the flexible tube, the hollow tube surrounding the flexible tube may be utilized as a heat sink or a heat source.

For example, the hollow tube may have a cavity or a lumen with an inner diameter matching or approximating the outer diameter of the flexible tube. In such a way, the flexible tube may be inserted into the cavity or lumen of the cavity or lumen such that a desired length of the tube is surrounded by the hollow tube. Accordingly, the hollow tube surrounding the flexible tube may impart density/flexibility differentials in the longitudinal, axial direction, and/or in the radial direction of the flexible tube. For example, the hollow tube may be heated to a temperature greater than or equal to approximately 250 degrees Celsius. In alternative embodiments, the hollow tube may be a heat sink, for example, to absorb heat from the tube as the flexible tube or mandrel(s) inserted within lumen(s) of the flexible tube is/are heated.

In some embodiments, the hollow tube may be made of one or more metals. The material comprising the hollow tube may be utilized to impart density/flexibility differentials or different characteristics on the flexible tube. For example, a first length of the hollow tube may comprise a first metal having a first conductance, and a second length of the hollow tube may comprise a second metal having a second conductance.

Accordingly, in some embodiments, as the flexible tube is heated or sintered, the hollow tube may absorb the heat from the flexible tube, and the different portions of the hollow tube may help to impart different characteristics to the flexible tube in a radial direction (e.g., within a cross-section of the flexible tube) and/or in a longitudinal direction (e.g., along an axial length of the flexible tube). For example, a first portion of the flexible tube within the first portion of the hollow tube may have different characteristics than a second portion of the flexible tube within the second portion of the hollow tube. For example, an outer portion of the flexible tube, or the portion of the flexible tube closer to the hollow tube may be less sintered, and thus have a lower density, than an inner portion of the flexible tube. In some embodiments, a first portion of the flexible tube within the first portion of the hollow tube may have different characteristics than a second portion of the flexible tube within the second portion of the hollow tube.

In alternative embodiments, the hollow tube may be heated to a temperature greater than or equal to approximately 250 degrees Celsius. In such a way, the hollow tube may be utilized to impart different characteristics to the flexible tube in a radial and/or longitudinal direction. For example, portions of the flexible tube closest to the hollow tube, or the heat source, may be more sintered, and thus have a higher density.

In further alternative embodiments, the density of the flexible tube in the radial and axial directions may be further controlled by inserting at least a portion of the flexible tube into the hollow tube and inserting one or more mandrels into the lumen(s) of the flexible tube, as described above. The cross-sectional density and/or flexibility of the flexible tube may be further controlled, for example, via inductively heating mandrels inserted through the lumens of the flexible tube. For example, one or more mandrels may be selectively placed inside one or more lumens of the flexible tube and inductively heated to suitable temperatures to create regions of varying density. In such a way, the one or more mandrels may be used as heat sources, and thus the desired characteristics may be imparted to the resulting flexible tube.

The one or more mandrels may be used in conjunction with, or in addition to, the hollow tube. For example, the hollow tube surrounding the flexible tube may be utilized as a heat sink to absorb heat from the flexible tube and/or mandrels. In such a way, the flexible tube may be heated and/or the one or more mandrels may be heated. Alternatively, the hollow tube may be used as a heat source. In such a way, the one or more mandrels may be used as heat sinks, and thus the desired characteristics may be imparted to the resulting flexible tube.

FIG. 7A shows a cross-section of an exemplary multi-lumen green tube 600A according to an embodiment, prior to processing steps according to this disclosure. Tube 600A includes various working channels or lumens 630a, 630b, 630c, and 630d, and four channels or lumens 630e used for control wires to control up, down, left, right steering. As an example shown in FIG. 7B, placing mandrels in only lumens 630b and 630c, and heating those mandrels, will create higher density in an area, such as area 610a, that may receive the greatest amount of heat and thereby reach the highest temperature. Area 610a may thereby have a higher density and lower flexibility than other portions of the cross section of the resulting tube 600B. Area 610a may extend along an axial length of tube 600B.

Similarly, placing mandrels in lumens 630a, 630d as well as 630b, 630c will result in a larger area 610b of material with a higher density and greater stiffness. Area 610b may thereby have a higher density and lower flexibility than other portions of the cross section of the resulting tube 600C. Area 610b may extend along an axial length of tube 600C.

As more mandrels are placed in additional lumens, the area of material with a higher density and greater stiffness increases. For example, placing mandrels in lumens 630e in addition to lumens 630a, 630b, 630c, 630d will result in a larger area 610c of higher density. Area 610c may extend along an axial length of tube 600D. Finally, placing mandrels in each of the lumens 630a-e and heating the mandrels sufficiently may result in an area 610d of higher density and lower flexibility. The area 610d may encompass most, if not all, of the entire cross section of the tube 600E.

The areas of higher density (610a-d) are not limited to those described. For example, the areas of higher density may be controlled, as desired, through the placement of mandrels in any combination of lumens 630a-e and may be nonconcentric. For example, if a user desired for the bottom portion of the tube to be more dense than the top portion, the user can insert and heat mandrels in the bottom lumens 630e and 630c. This will create a density gradient in the cross-sectional portion of the tube with an area of higher density near the bottom, adjacent to at least the lumens 630e and 630c.

Furthermore, the areas of relatively higher density may be controlled by the relative thermal conductances and magnetic inductances of the mandrels (e.g., according to mandrel material) and/or the temperatures of the heated mandrels. For example, heating a mandrel to a first temperature will result in a first area of higher density, and heating a mandrel to a second, higher temperature will result in a second, larger area of higher density. The axial length of the areas of higher density may be controlled by the length of the mandrel.

A wide variety of materials may be used for the mandrels and/or hollow tube, depending on the desired characteristics to be imparted on the flexible tube. For example, tungsten, bronze, brass, copper, silver coated copper, Inconel, nitinol, various stainless steels (302, 304, 316, 420, etc.), as well as clad materials may be used. Further, different geometries of these materials may also be used.

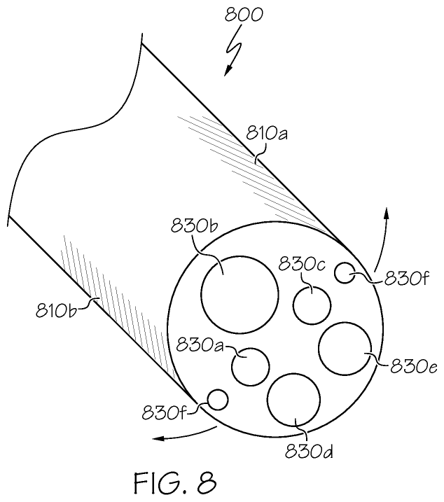

FIG. 8 depicts a distal end portion of an exemplary multi-lumen tube 800 with variable stiffness. FIG. 8 demonstrates how an articulation joint may be created by the insertion of the mandrels in selected lumens to create preferred bending regions. For example, in this embodiment, an articulation joint having two-way bending can be created by placing mandrels in lumens 830b, 830d, and 830e and heating the mandrels, as previously described. This processing creates a tube 800 with at least two regions 810a, 810b, of decreased densities on either side of tube 800 relative to the top and bottom portions (i.e. the areas adjacent to where the mandrels were placed). These at least two regions may have densities less than the remaining circumference. For example, the circumferences between the two regions may have a greater density than the at least two regions. The at least two regions may be separated about the circumference of the tube by approximately 180 degrees in order to accomplish two-way bending in opposite directions, as shown. However, these regions may be separated by a varying degree to accomplish any desired direction of steering (i.e. left and down, left and up, right and down, or right and up). The axial length of the portions may be controlled by varying the length of the mandrel. Regions 810a, 810b of decreased densities, and therefore greater flexibility, are adjacent lumens 830f that house controls wires that are translated within those lumens 830f to cause bending in the directions of the arrows shown in FIG. 8. Those bending directions are in the plane of the regions 810a, 810b of greater flexibility, assisting in the bending operation. It may be understood that the creation of two regions 810a, 810b of decreased density is not limited to the scope of this disclosure. For example, more or less areas of decreased or increased densities can be made. The creation of the areas of increased or decreased density is dependent on the spacing and orientation of the lumens in which mandrels are inserted.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device, comprising:
a shaft, wherein the shaft includes an integral tube defining a plurality of lumens, the integral tube comprised of at least one of PTFE and ePTFE, and wherein a density of the at least one of PTFE and ePTFE varies along a length of the integral tube.

2. The medical device of claim 1, wherein the density varies within a cross-section of the integral tube.

3. The medical device of claim 2, wherein a density of a radially inner portion of the integral tube differs from a density of a radially outer portion of the integral tube.

4. The medical device of claim 1, wherein the integral tube includes an articulation joint, and wherein at least two regions separated about a circumference of the articulation joint have densities less than other regions about the circumference.

5. The medical device of claim 4, wherein the at least two regions are separated about the circumference by approximately 180 degrees.

6. The medical device of claim 1, wherein the integral tube comprises an articulation joint, and the integral tube further includes a portion proximal to the articulation joint, wherein the articulation joint is less dense than the proximal portion.

7. The medical device of claim 1, further comprising one of a braid or a coil surrounding an outermost surface of the integral tube.

8. The medical device of claim 7, wherein the one of the braid or the coil is at a temperature of at least 250 degrees Celsius.

9. The medical device of claim 7, wherein at least one of a pitch, a material, and a thickness of a wire of the one of the braid and the coil varies along a length of the one of the braid and the coil.

10. The medical device of claim 1, wherein the integral tube further comprises at least one mandrel extending through at least one of the plurality of lumens.

11. The medical device of claim 10, wherein the at least one mandrel is at a temperature greater than or equal to 250 degrees Celsius.

12. The medical device of claim 1, wherein the integral tube further comprises a plurality of mandrels extending through more than one lumen of the plurality of lumens.

13. The medical device of claim 12, wherein a first mandrel of the plurality of mandrels is at a first temperature, and a second mandrel of the plurality of mandrels is at a second temperature different from the first temperature.

14. The medical device of claim 1, wherein a density of a proximal portion of the integral tube differs from a density of a distal portion of the integral tube.

15. The medical device of claim 1, wherein the medical device is an endoscope, the shaft is connected to a handle at a proximal end of the shaft, and the plurality of lumens include a working channel and lumens for articulation wires.

16. A method of fabricating a tube, the method comprising:
stretching a heated, integral starting tube of at least one of PTFE and ePTFE, to result in an intermediate tube having a density less than a density of the integral starting tube, wherein the starting tube defines multiple lumens; and
linearly retracting only a first portion of a length of the intermediate tube when the intermediate tube is heated, to result in a final tube, wherein the first portion has a greater density than a second portion of the final tube.

17. The method of claim 16, further comprising:
inserting a mandrel through a lumen of the multiple lumens;
heating the mandrel to at least 250 degrees Celsius; and
removing the mandrel.

18. The method of claim 16, further comprising:
applying tension to at least one of the starting tube, the intermediate tube, and the final tube.

19. The method of claim 17, further comprising:
controlling the linear retraction by applying a force in a longitudinal direction of the tube.

20. A method of forming an articulation joint, the method comprising:
inserting at least one mandrel through at least one lumen of a multi-lumen tube comprising at least one of PTFE and ePTFE;
heating the mandrel to a temperature of at least 250 degrees Celsius to result in a first region of the tube having a higher density than a second region of the tube, the first region and the second region being in a same cross-section of the tube; and
removing the at least one mandrel.
